(19) United States
(12) Patent Application Publication
(10) Pub. No.: US 2021/0302407 A1
(43) Pub. Date: Sep. 30, 2021

(54) Quality Control Method for Moulded Plastics Components

(71) Applicant: Norton (Waterford) Limited, Waterford (ID)

(72) Inventors: Daniel Buck, Waterford (IE); Declan Walsh, Waterford (IE); Paul Prendergast, Waterford (IE)

(73) Assignee: Norton (Waterford) Limited, Waterford (IE)

(21) Appl. No.: 16/482,700

(22) PCT Filed: Jan. 30, 2018
(86) PCT No.: PCT/EP2018/052250

§ 371 Date: Jul. 31, 2019

(30) Foreign Application Priority Data
Feb. 1, 2017 (GB) 1701669.2

(51) Int. Cl. G01N 33/44; G01N 30/02

(52) U.S. Cl. CPC G01N 33/442; G01N 2030/025; G01N 30/02

(57) ABSTRACT

Disclosed herein is a quality control method for predicting premature degradation in mechanical properties of a series of moulded plastics components, the moulded plastics components being formed from a batch mixture comprising a cross-linkable polymer and a peroxide cross-linking agent. The method comprises selecting a subset of the series of moulded plastics components and obtaining a material specimen from the subset; and then extracting and measuring a residual peroxide content of the specimen. According to the invention, the measured residual peroxide content is representative of a probability of the pre-measuring mature age-related degradation in the entire series of moulded plastics components, and may therefore be compared against a threshold residual peroxide content to determine whether the entire series of components is of acceptable quality.

### CROSS-REFERENCE TO RELATED APPLICATIONS

This application claims the benefit of GB Patent Application No. GB 1701669.2 filed Feb. 1, 2017 entitled “Quality Control Method for Moulded Plastics Components” and the benefit of PCT Patent Application No. PCT/EP2018/052250 filed Jan. 30, 2018 entitled “Quality Control Method for Moulded Plastics Components,” which are incorporated by reference herein in their entireties.

### FIELD OF THE INVENTION

This invention relates to a quality control method for predicting premature age-related degradation in mechanical properties of moulded plastics components formed from a mixture comprising a cross-linkable polymer and a peroxide cross-linking agent. The moulded plastics components may be elastomeric seals for use in the metering valve of a pressurised medicament container, such as is used in a pressurised metered dose inhaler (pMDI).

The invention also relates to a method of manufacturing the series of moulded plastics components, wherein the manufacturing method includes the quality control method.

### BACKGROUND OF THE INVENTION

Pressurised metered dose inhalers (pMDIs) provide a convenient means for the self-treatment of a variety of diseases, including respiratory diseases such as asthma and COPD (chronic obstructive pulmonary disease).

A pressurised metered dose inhaler typically comprises a substantially-cylindrical container in which a mixture of medicament in solution or suspension-form together with a propellant is stored under pressure. One end of the container is provided with a metering valve having a tubular valve stem which can be displaced longitudinally to selectively dispense a metered amount of the medicament formulation. The propellant serves to atomise the medicament upon dispensation, with the resulting plume of medicament then being inhaled deep into the patient's respiratory tract. The inhaler also comprises an actuator having a cavity for receiving the metering-valve end of the container, a so-called stem block for longitudinally-displacing the valve stem relative to the container when the container is pressed into the housing, and a mouthpiece through which the dispensed medicament is directed for inhalation by the patient.

An important aspect of the performance of a pressurised metered dose inhaler is its ability store the medicament formulation in a stable manner for extended periods of time (i.e. up to several years) and in a variety of ambient conditions (i.e. fluctuating and extreme temperatures and pressures), including the prevention of leakage of the formulation from the container. Such leakage can cause an inhaler to unexpectedly run out of medicament before the expected number of doses has been dispensed. Medicament leakage can also lead to inaccurate or inconsistent dosing, for example if the leakage is from a metering chamber of the metering valve.

In recent years significant efforts have been made to improve the design of pressurised metered dose inhalers. Developments have included new actuators in which the design of the medicament flow path, particularly the stem block and/or mouthpiece, has been refined to improve consistency in the delivered dose. So-called breath-actuated variants have also been developed for use by patients that struggle with the co-ordination needed for effective use of a manually-actuated inhaler.

New metering valves for the pressurised medicament containers have also been developed, including so-called primeless metering valves in which the medicament formulation is able to flow more freely between the bulk container and a metering chamber of the valve prior to actuation. A known primeless metering valve is disclosed in WO 2006/021797 A1, the entire contents of which is incorporated herein by reference. Medicament containers having primeless metering valves have been found to be particularly suitable for use with breath-actuated inhalers, since in such inhalers it can be difficult to prime the metering valve without inhaling medicament.

The material composition of metering valve components, such as the seals which bear against the longitudinally-displaceable valve stem, has also received attention in the prior art.

Although significant progress has been made, there remains a need for pressurised metered dose inhalers having improved performance, especially in relation to their ability to stably store the medicament formulation and deliver a consistent dose. There is also a need for techniques and methods for facilitating the manufacture of such inhalers.

### SUMMARY OF THE INVENTION

The inventors have discovered that known pressurised metered dose inhalers are particularly susceptible to leakage of the medicament formulation when they are stored and/or used in relatively high temperature conditions (e.g. 40° C.) for extended periods of time (e.g. six months). The inventors have also discovered that such leakage is usually caused by failure of the seals within the medicament container, in particular those within the metering valve, when the inhaler is actuated. Failure of the metering valve seals in this way is referred to as a “blow-by” failure, since the medicament formulation passes between the seal and the outer surface of the valve stem against which the seal bears. Surprisingly, it has now been discovered that such blow-by failures are frequently not attributable to the design of the seals or the surrounding structure, but rather the quality of the elastomeric material from which they are formed.

The invention provides a quality control method for predicting premature degradation in mechanical properties of a series of moulded plastics components, the moulded plastics components being formed from a batch mixture comprising a cross-linkable polymer and a peroxide cross-linking agent, the method comprising: selecting a subset of the series of moulded plastics components and obtaining a material specimen from the subset; and extracting and measuring a residual peroxide content of the specimen, wherein the measured residual peroxide content is representative of a probability of the premature age-related degradation in the entire series of moulded plastics components.

The invention is based on the insight that blow-by failures in the seals of pressurised metered dose inhalers are partly attributable to poor mechanical properties of the seal material caused by under-cross-linking (i.e. incomplete curing) of the cross-linkable polymer, for example EPDM (ethylene propylene diene). This under-cross-linking of the polymer leads to reduced cross-linking density and/or cross-linking consistency. Furthermore, it has been recognised that under-cross-linking of the polymer will inevitably be accompanied by incomplete consumption of the peroxide cross-linking agent, which agent remains in the material and may then induce premature degradation of the polymer by chain-scission, especially at elevated temperatures.

The inventors have recognised that seal material quality, in particular the material's susceptibility to degradation in mechanical properties of the seals, can therefore be assessed by obtaining a specimen of the seal material, and extracting and measuring the residual peroxide content in the specimen. A higher measured residual peroxide content correlates with a greater probability of premature degradation, and therefore premature seal failure and leakage of medicament. The measured residual peroxide content can accordingly be used as a measure of material quality.

Although the quality control method of the invention is considered to be particularly suitable for use in the manufacture of elastomeric seals for the metering valves of pressurised medicament canisters, it is also suitable for assessing the material quality of any moulded plastics components that are formed from a batch mixture comprising a cross-linkable polymer and a peroxide cross-linking agent.

Since the quality control method of the invention is destructive of the components tested, it is carried out on a subset of components taken from a batch of manufactured components. The subset may, for example, comprise one, five or ten components, the results for which are taken to be representative of the entire batch.

In a specific embodiment, the quality control method further comprises the step of comparing the measured residual peroxide content to a threshold residual peroxide content. The threshold may correspond to a residual peroxide content above which the risk or probability of premature degradation is considered to be unacceptable. The threshold value may be determined experimentally by manufacturing and testing a series of the components having different residual peroxide contents.

Since the measured residual peroxide content is considered to be representative of the whole series of moulded plastics components, the method may further comprise the step of setting-aside (e.g. scrapping) the entire series if the measured residual peroxide content exceeds the threshold residual peroxide content. The set-aside components may be disposed of, recycled or diverted for some other, less-demanding use.

In certain embodiments, the moulded plastic components may comprise a cross-linked elastomer, particularly EPDM (ethylene propylene diene). The moulded plastic components may further comprise one or more plasticisers and/or fillers, as is known generally in the art. In a preferred embodiment, the moulded plastics components are elastomeric seals for use in pressure vessels, and in a particularly preferred embodiment, the seals are elastomeric seals for use in a metering valve of a pressurised medicament canister.

According to the invention, the residual peroxide content may be extracted from the specimen by a solvent extraction technique, for example using acetone as the solvent. The solvent extraction process may be accelerated, for example using heat and/or pressure. Additionally or alternatively, the specimen may be finely divided, for example by mechanically milling, to accelerate the solvent extraction process.

According to the invention, at least one peroxide compound may then be isolated by chromatography, for example gas chromatography, and the at least one peroxide compound may be identified and its amount quantified by mass spectrometry. In a specific embodiment, the isolation, identification and quantification of the peroxide compound may be carried out on an integrated gas chromatography mass spectrometry (GCMS) detector.

The residual peroxide content on which the invention is based may relate to the residual amount(s) of at least one peroxide compound selected from 1,3 bis-[tert-butylperoxyisopropyl]benzene; 1,4 bis-[tert-butylperoxyisopropyl]benzene; 1,3 isopropanol-tert-butylperoxyisopropylbenzene; 1,4 isopropanol-tert-butylperoxyisopropylbenzene; 1,3 acetyl-tert-butylperoxyisopropylbenzene; and 1,4 acetyl-tert-butylperoxyisopropylbenzene.

It has been found that the presence and/or measured amount of each of these peroxide compounds correlates with susceptibility to premature degradation in the mechanical properties of the components. A threshold residual peroxide content for one or more of these compounds may be 40 μg/g or less, for example 35 μg/g or less, or even 30 μg/g or less.

A further aspect of the invention provides a method of manufacturing a series of moulded plastics components comprising: providing a materials batch comprising measured amounts of a cross-linkable polymer and a peroxide cross-linking agent, and, optionally, one or more plasticisers and/or fillers; homogenising the materials batch by mixing to form a batch mixture; moulding the batch mixture into at least one shape, particularly by extruding the batch mixture into a plurality of elongated strips; curing the moulded shape by applying heat and/or pressure to cause cross-linking of the cross-linkable polymer by the peroxide cross-linking agent; and punching or cutting out moulded plastics components from the moulded shape after curing, wherein the method further comprises the quality control method described above.

The manufacturing method may further comprise modifying one or more mixing parameters (e.g. time, speed, etc.) of the homogenising step and/or modifying heat and/or pressure conditions (e.g. time, temperature, pressure, etc.) of the curing step in dependence on the measured residual peroxide content. Such a method provides closed-loop feedback control of the material quality.

In a specific embodiment of the manufacturing method, the moulded plastics components are elastomeric seals, and the method further comprises assembling the elastomeric seals into metering valves for pressurised medicament containers. In a particularly preferred embodiment, the metering valves are assembled into a breath-actuated pressurised metered dose inhaler.

### BRIEF DESCRIPTION OF THE DRAWINGS

Specific embodiments of the invention will now be described in greater detail, with reference to the accompanying drawings, in which:

FIG. 1 is a plot illustrating the mechanical properties of cross-linked EPDM (ethylene propylene diene);

FIG. 2 is a flow chart illustrating the manufacturing process for moulded EPDM seals for use in a metering valve;

FIG. 3 is a flow chart illustrating the quality control method according to an embodiment of the invention; and

FIGS. 4a and 4b are charts illustrating the results of residual peroxide analysis of the examples.

### DETAILED DESCRIPTION

The invention will now be described in the context of a specific embodiment involving elastomeric seals for use in a metering valve of a pressurised medicament container. However, the quality control method of the invention is not limited to use with such components and may in principle be used with any moulded plastics components that are formed from a batch mixture comprising a cross-linkable polymer and a peroxide cross-linking agent.

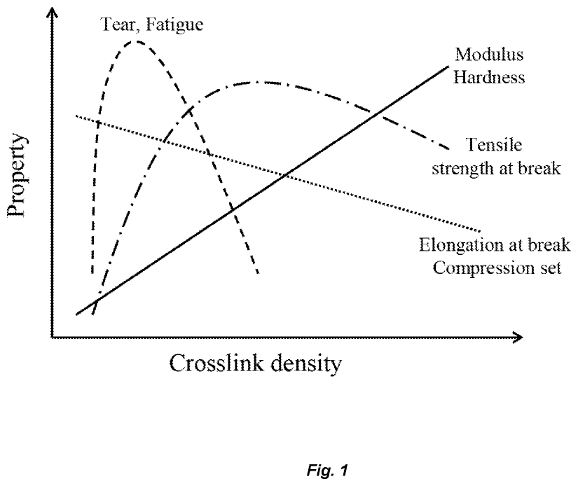

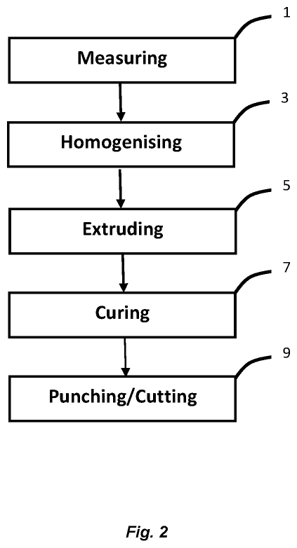

A known metering valve for a pressurised medicament container is disclosed in FIGS. 1 and 2 of WO 2006/021797 A1. The metering valve disclosed in this document is a primeless valve, which is therefore particularly suitable for use in a breath-actuated inhaler. The metering valve comprises a tubular valve stem which can be longitudinally-displaced against a spring bias. An annular metering chamber is arranged about the valve stem. When the valve stem is in its rest position, the metering chamber is in fluid communication with the bulk medicament formulation stored in the container, from which it is filled. When the valve stem is longitudinally displaced, the fluid communication between the metering chamber and the bulk formulation is initially cut-off, with further longitudinal displacement then opening a fluid path between the metering chamber and the interior of the valve stem. A metered dose of the medicament is thereby dispensed from an open end of the valve stem.

The annular metering chamber of the metering valve is defined by a rigid plastics component having a substantially-cylindrical inner wall, through which the valve stem extends. Elastomeric seals, or valve seats, are mounted at opposite longitudinal ends of the metering chamber and bear in a radially-inward direction against the outer surface of the valve stem. A first, inner valve seat seals the metering chamber from the bulk formulation stored in the container and a second, outer valve seat seals the metering chamber from the external environment. Both valve seats form a dynamic sliding seal. The second valve seat of such a metering valve has been found to be particularly susceptible to blow-by failures, which failures cause leakage of the medicament formulation from the metering chamber, and thereby inconsistencies in the amount of the delivered dose. Blow-by failures involve the momentary loss of contact between the valve seat and the valve stem.

The seals of the metering valve described above are formed of an elastomer composition comprising EPDM (ethylene propylene diene), together with a stearic acid plasticiser and magnesium silicate and precipitated silica fillers. The EPDM is provided as an uncross-linked base material having a high ethylene content, which can be moulded into a desired shape. The stearic acid plasticiser aids in processability of the uncross-linked material, whereas the fillers improve the strength and toughness of the elastomer.

The EPDM in this form is highly amorphous and requires cross-linking in order to achieve the mechanical properties needed for sealing applications. Cross-linking is the process of chemically producing network junctions by the insertion of cross-links between the polymer chains. Cross-linking is achieved by adding a peroxide cross-linking agent to the elastomer composition. In particular, thermal decomposition of the peroxide cross-linking agent during and after the moulding process yields free radicals which provide active sites for cross-linking on the backbone of the EPDM base material.

EPDM is a terpolymer built from monomers of ethylene, propylene, and a diene-component. Suitable diene-components are for example dicyclopentadiene (DCPD), ethylidene norbornene (ENB), and vinyl norbornene (VNB) yielding ethylene-propylene-dicyclopentadiene copolymer (CAS Reg. No. 25034-71-3), ethylene-propylene-ethylidenenorbornene copolymer (CAS Reg. No. 25038-36-2) and ethylene-propylene-vinylnorbornene copolymer (CAS Reg. No. 27155-45-9). The “M” in EPDM refers to its classification in ASTM standard D-1418 denoting polymers having a saturated backbone chain of the poly-methylene type. An idealized generic structure of EPDM with ENB as diene component is provided in formula (I) below. The dienes serve as sites of cross-linking. The properties of the EPDM depend e.g. on the content of each component, the type of diene, dispersity as well as on the degree of cross-linking.

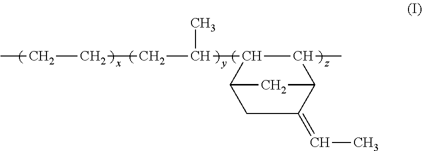

The effects of cross-linking density on the mechanical properties of the elastomer are illustrated in FIG. 1. It will be seen that all of the properties vary with cross-linking density. It will be seen in particular that compression set, which negatively affects sealing performance, is inversely proportional to cross-linking density. Compression set of an elastomer is the amount of permanent deformation which remains when a force applied to the elastomer is removed.

In general, the peroxide cross-linking agent is added to the elastomer composition in an amount such that it will be entirely consumed in the cross-linking process, with the resulting elastomer having a sufficiently high cross-linking density. Since peroxide is highly active, any residual (unconsumed) peroxide remaining after the cross-linking process can lead to premature degradation of the elastomer by chain-scission. The elastomer is particularly susceptible to such premature degradation when stored for extended periods of time and/or at elevated storage temperatures.

FIG. 2 illustrates, as a flow chart, a method of manufacturing a batch of the seals of the metering valve described above. In a first step 1, a batch of materials is provided. The batch of materials comprises precisely-measured amounts of uncross-linked EPDM, a peroxide cross-linking agent, a stearic acid plasticiser, and magnesium silicate and precipitated silica fillers.

In a second step 3 of the method, the materials batch is homogenised by mixing in a mixer to form a batch mixture. The batch mixture is further homogenised by transferring it to an open two roll mill for milling, to provide an even distribution of all constituents.

In a third step 5 of the method, the homogenised uncross-linked compound is transferred to an extrusion apparatus in which it is formed into a plurality of thin, elongate strips.

In a fourth step 7 of the method, the moulded strips are laid in a compression mould and heat and pressure are applied to initiate curing of the EPDM compound. The curing process involves decomposition of the peroxide cross-linking agent, which causes cross-linking of the EPDM. The moulded strips are removed from the compression mould as well-defined strips having a consistent thickness, and are post-mould cured in a convection oven to complete the cross-linking process.

In a fifth step 9 of the method, the cross-linked EPDM strips are fed into an automated punch in which annular-shaped seals are punched-out. This step of the method yields a batch of several hundred or even several thousand individual seals, which are then washed in ethanol to remove reaction products left over from the manufacturing process before being inspected and assembled into metering valves.

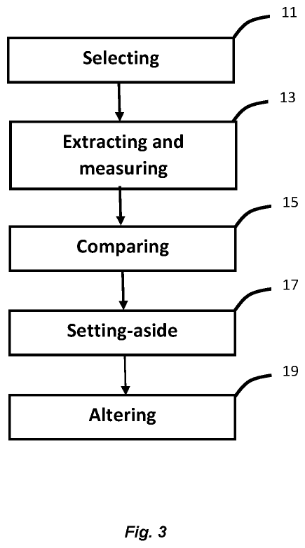

The invention provides a quality control method for predicting premature degradation in mechanical properties of the batch of seals. FIG. 3, illustrates, as a flow chart, an embodiment of the quality control method. In a first step 11, a subset of five seals is selected from the batch of seals manufactured in the method described with reference to FIG. 2. A nominal 1.0 g specimen of elastomer is then cut-out from each seal.

In a second step 13, a residual peroxide content of the specimen is extracted and measured, with the measured residual peroxide content being representative of a probability of the premature age-related degradation in the entire series of moulded plastics components.

The residual peroxide is extracted from the specimen by an accelerated solvent extraction (ASE) technique which uses acetone as a solvent to extract the peroxide at an elevated temperature and pressure.

In a particular embodiment of the invention the residual peroxide content is extracted using an Accelerated Solvent Extraction (ASE) method, e.g. with a Dionex ASE system or equivalent.

In a particular embodiment of the invention the ASE is performed at about 80° C. and about 1000 psi.g (corresponding to about 69 bar).

The acetone solvent containing the extracted peroxide is then subjected to analysis by a gas chromatography mass spectrometry (GCMS) technique. In particular, at least one peroxide compound is isolated by gas chromatography, which compound is then identified and quantified by mass spectrometry. The use of gas chromatography mass spectrometry techniques and equipment is well-known to those of ordinary skill in the art.

In one embodiment, the chromatograph is an Agilent 5890, 6890, 7890 or equivalent gas chromatograph and the detector is an Agilent 5971, 5972, 5973, 5975 El quadrupole or equivalent mass selective detector (MSD). In one embodiment the carrier gas is helium. In one embodiment the column operating temperature is about 325° C. In one embodiment the MS quadrupole temperature is about 150° C. In one embodiment the MS source temperature is about 230° C.

The gas chromatography mass spectrometry technique described above is used in particular to isolate, identify and quantify residual amounts of the following peroxide compounds in the specimen:
1,3 bis-[tert-butylperoxyisopropyl]benzene,
also denoted 1,1′-[1,3-phenylenebis(1-methylethylidene)]bis[2-(1,1-dimethylethyl) peroxide],
CAS Reg. No. [2212-81-9],
denoted herein “1,3 Intact Peroxide”,
1,3 isopropanol-tert-butylperoxyisopropylbenzene,
also denoted 3[1-[(1,1-dimethylethyl)dioxy]-1-methylethyl]-α,α-dimethylbenzenemethanol,
CAS Reg. No. [57913-37-8],
denoted herein “1,3 Half-Peroxide A”;
1,4 isopropanol-tert-butylperoxyisopropylbenzene,
also denoted 4-[1-[(1,1-dimethylethyl)dioxy]-1-methylethyl]-α,α-dimethylbenzenemethanol,
CAS Reg. No. [57807-44-0],
denoted herein “1,4 Half-Peroxide A”;
1,3 Acetyl-tert-butylperoxyisopropylbenzene,
also denoted 1-[3-[1-[(1,1-dimethylethyl)dioxy]-1-methylethyl]phenyl]ethanone,
CAS Reg. No. [137489-71-5],
denoted herein “1,3 Half-Peroxide B”; and
1,4 Acetyl-tert-butylperoxyisopropylbenzene,
also denoted 1-[4-[1-[(1,1-dimethylethyl)dioxy]-1-methylethyl]phenyl]ethanone,
CAS Reg. No. [79506-32-4],
denoted herein “1,4 Half-Peroxide B”.

It is also possible to isolate, identify and quantify residual amounts of 1,4 bis-[tert-butylperoxyisopropyl]benzene (also denoted 1,1′-[1,4-phenylenebis(1-methylethylidene)]bis[2-(1,1-dimethylethyl) peroxide], CAS Reg. No. [2781-00-2], denoted herein “1,4 Intact Peroxide”). The residual amounts of peroxide are specified in μg of peroxide per g of EPDM elastomer (μg/g).

In a third step 15 of the method, the measured residual amounts of peroxide compounds are compared to a threshold residual peroxide content. Examples of suitable threshold residual peroxide contents are provided below in Table 1 which represent residual peroxide contents above which the risk of premature degradation in mechanical properties of the seals is considered to be unacceptably high. Suitable thresholds can, for example, be determined by producing seals having different residual peroxide contents and then subjecting the seals to testing over an extended period of time.

In one embodiment, the residual peroxide content on which the invention is based relates to the residual amount(s) of at least one peroxide compound selected from 1,3 Intact Peroxide; 1,4 Intact Peroxide; 1,3 Half-Peroxide A; 1,4 Half-Peroxide A; 1,3 Half-Peroxide B; and 1,4 Half-Peroxide B.

In one embodiment, the residual peroxide content on which the invention is based relates to the residual amount(s) of at least one peroxide compound selected from 1,3 Intact Peroxide; 1,3 Half-Peroxide A; 1,4 Half-Peroxide A; 1,3 Half-Peroxide B; and 1,4 Half-Peroxide B.

In one embodiment, the residual peroxide content on which the invention is based relates to the residual amount(s) of 1,3 Half-Peroxide B and/or 1,4 Half-Peroxide B.

In one embodiment, the residual peroxide content on which the invention is based relates to the sum of the residual amounts of 1,3 Intact Peroxide; 1,3 Half-Peroxide A; 1,4 Half-Peroxide A; 1,3 Half-Peroxide B; and 1,4 Half-Peroxide B.

In one embodiment, the threshold residual peroxide content for the sum of all measured peroxide compounds is 40 μg/g or less, particularly 35 μg/g or less, most particularly 30 μg/g or less.

In one embodiment, the threshold residual peroxide content for 1,3 Half-Peroxide B is 20 μg/g or less, particularly 16 μg/g or less, most particularly 12 μg/g or less.

In one embodiment, the threshold residual peroxide content for 1,4 Half-Peroxide B is 20 μg/g or less, particularly 16 μg/g or less, most particularly 12 μg/g or less.

TABLE 1
Threshold contents for residual peroxides.

| | Threshold residual peroxide |
|---|---|
| Peroxide compound | content [μg/g] |
| 1,3 Intact Peroxide | 1 |
| 1,4 Intact Peroxide | 1 |
| 1,3 Half-Peroxide A | 1 |
| 1,4 Half-Peroxide A | 1 |
| 1,3 Half-Peroxide B | 20 |
| 1,4 Half-Peroxide B | 20 |
| overall residual peroxides | 40 |

In a fourth step 17 of the method, the entire batch of seals is set-aside if the measured residual peroxide content exceeds the threshold residual peroxide content. In particular, the seals are not assembled into metering valves and may be scrapped.

In a fifth step 19 of the method, the mixing parameters of the homogenising step 5 and the heat and/or pressure conditions of the curing step 7 of the seal manufacturing method are altered in dependence on the measured residual peroxide content. For example, in the event that the measured residual peroxide content exceeds the threshold value, a subsequent batch mixture can be milled for longer to improve the homogeneity of the mixture and/or the period of time for which the seals are cured can be extended.

Although a specific embodiment has been described above, various modifications may be made without departing from the scope of the invention, which is defined by the claims.

For example, in the embodiment described above, the moulded plastics components are valve seats. The method of the invention may, however, be applied to other moulded components, but is particularly suited to moulded elastomeric components such as seals.

In the embodiment described above the residual peroxide content is isolated, identified and quantified using the GCMS technique. However, other known techniques, such as iodometric titration, may be used.

The third, fourth and fifth steps 15, 17, 19 of the embodiment shown in FIG. 3 are optional.

### Example 1

A batch of EPDM valve seals, or seats, of the type used in a metering valve for a pressurised medicament container were produced using the method described above with reference to FIG. 2.

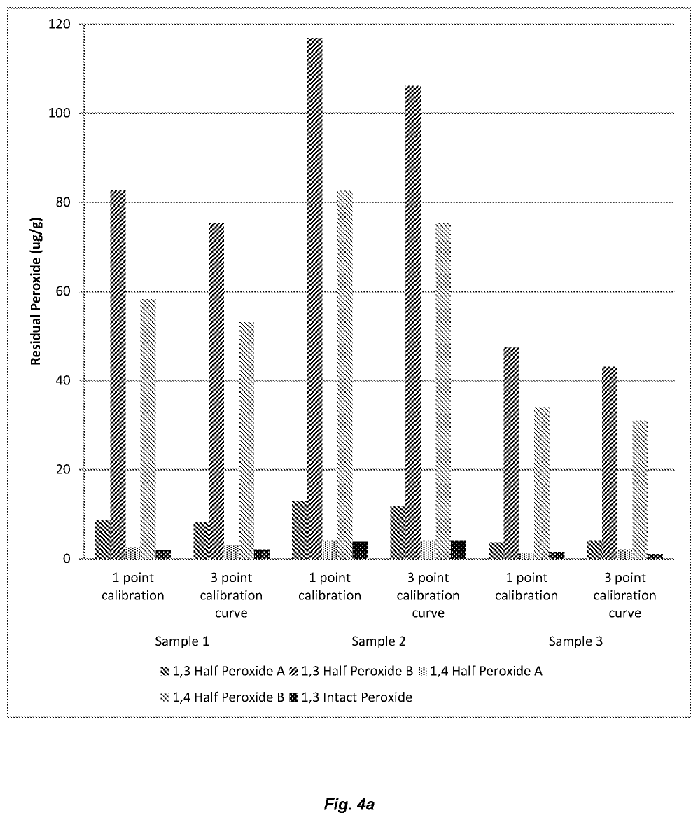

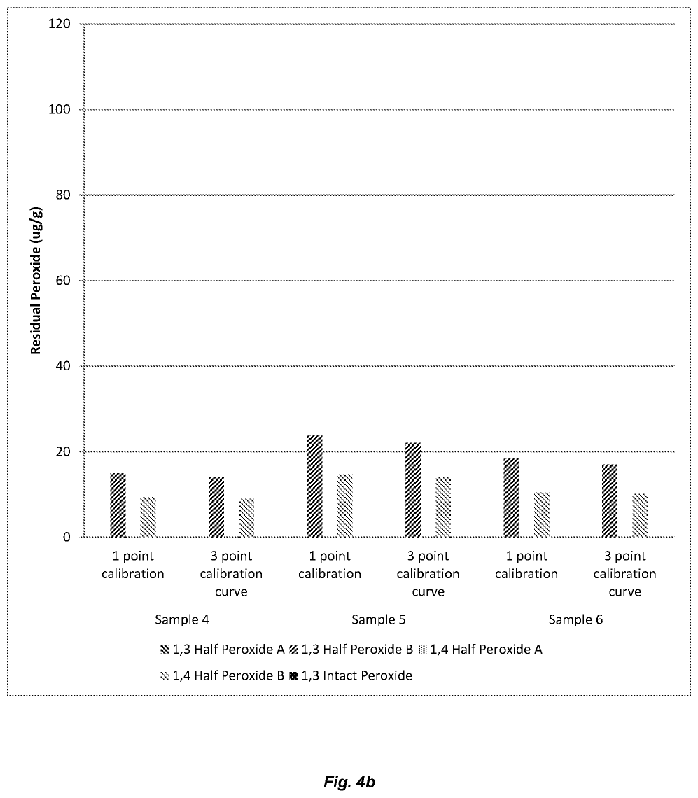

A subset of three of the seals was selected and subjected to the quality control method described above with reference to FIG. 3 (specimens 1 to 3). The results of the gas chromatography mass spectrometry (GCMS), adopting one and three point calibration techniques, on the three samples are illustrated in FIG. 4a.

### Example 2

The manufacture of seals according to example 1 was repeated except that, in the process for producing the valve seats, the second step 3 was modified by doubling the period of time for which the batch mixture was milled in the open two roll mill. Further, in the fourth step 7 of the method, the period of time for which the moulded strips remained in the compression mould was increased from 5.5 minutes to 7.5 minutes, and the post-mould curing time was increased from 6.0 minutes to 7.5 minutes. These measures were taken to improve the homogeneity of the batch mixture and to improve cross-linking density in the curing process.

A subset of three of the seals was selected and subjected to the quality control method described above with reference to FIG. 3 (specimens 4 to 6). The results of the gas chromatography mass spectrometry (GCMS) on the three samples are illustrated in FIG. 4b.

It can be seen by comparing FIGS. 4a and 4b that the residual peroxide content of the seals of example 2, having improved material homogeneity and cross-linking density, were significantly less than that of the seals of example 1. In this way, the quality control method of the invention provides an effective means for predicting premature degradation in mechanical properties of the seals.

In both, examples 1 and 2, the residual peroxide content was extracted using an Accelerated Solvent Extraction (ASE) method using a Dionex ASE system with acetone as solvent performed at 80° C. and 1000 psi.g (corresponding to about 69 bar).

In both, examples 1 and 2, the GCMS analysis was performed with an Agilent 5890, 6890, or 7890 GC and an Agilent 5971, 5972, 5973, or 5975 El quadrupole mass selective detector (MSD) using helium as carrier gas, a column operating temperature of 325° C., electron ionization, an MS Resolution/Dwell Time High/100, an MS quadrupole temperature of 150° C., and an MS source temperature of 230° C.

In both, examples 1 and 2, the one point calibration and three point calibration were conducted in the same manner but to different levels of detection (LOD). The one point calibration curve was conducted to an LOD at the parts per billion (ppb) level and the three point calibration to an LOD at the parts per million (ppm) level. The calibration standard for both was prepared to different concentrations for each reference compound to be detected.

Values were obtained by calculating a concentration ratio and a response ratio for each level for that compound. The concentration ratio corresponds to the concentration of the detected compound divided by the concentration of the internal standard for a given calibration level. The response ratio corresponds to the response of the detected compound divided by the response of the internal standard at that level. The software employed can calculate a calibration curve which may be than used to determine the result in μg/g.

1. A quality control method for predicting premature degradation in mechanical properties of a series of moulded plastics components, the moulded plastics components being formed from a batch mixture comprising a cross-linkable polymer and a peroxide cross-linking agent, the method comprising:
selecting a subset of the series of moulded plastics components and obtaining a material specimen from the subset; and
extracting and measuring a residual peroxide content of the specimen,
wherein the measured residual peroxide content is representative of a probability of the premature age-related degradation in the entire series of moulded plastics components.

2. The quality control method of claim 1, further comprising the step of comparing the measured residual peroxide content to a threshold residual peroxide content.

3. The quality control method of claim 2, further comprising the step of setting-aside the entire series of moulded plastics components if the measured residual peroxide content exceeds the threshold residual peroxide content.

4. The quality control method of claim 1, wherein the moulded plastic components comprise a cross-linked elastomer.

5. The quality control method of claim 4, wherein the moulded plastic components further comprise one or more plasticisers and/or fillers.

6. The quality control method of claim 1, wherein the moulded plastics components are elastomeric seals for use in pressure vessels.

7. The quality control method of claim 6, wherein the elastomeric seals are configured for use in a metering valve of a pressurised medicament canister.

8. The quality control method of claim 1, wherein the residual peroxide content is extracted from the specimen by solvent extraction.

9. The quality control method of claim 8, wherein at least one peroxide compound is isolated by chromatography.

10. The quality control method of claim 9, wherein said measuring the residual peroxide content of the specimen comprises identifying and quantifying the at least one peroxide compound by mass spectrometry.

11. The quality control method of claim 9, wherein the at least one peroxide compound includes at least one of 1,3 bis-[tert-butylperoxyisopropyl]benzene; 1,3 isopropanol-tert-butylperoxyisopropylbenzene; 1,4 isopropanol-tert-butylperoxyisopropylbenzene; 1,3 acetyl-tert-butylperoxyisopropylbenzene and 1,4 acetyl-tert-butylperoxyisopropylbenzene, particularly 1,3 acetyl-tert-butylperoxyisopropylbenzene and/or 1,4 acetyl-tert-butylperoxyisopropylbenzene.

12. The quality control method of claim 10, wherein the quantified residual peroxide content is the sum of the residual amounts of 1,3 bis-[tert-butylperoxyisopropyl]benzene; 1,3 isopropanol-tert-butylperoxyisopropylbenzene; 1,4 isopropanol-tert-butylperoxyisopropylbenzene; 1,3 acetyl-tert-butylperoxyisopropylbenzene; and 1,4 acetyl-tert-butylperoxyisopropylbenzene.

13. The quality control method of claim 2, wherein the threshold residual peroxide content is 40 μg/g or less.

14. A method of manufacturing a series of moulded plastics components, the method comprising:
providing a materials batch comprising measured amounts of a cross-linkable polymer and a peroxide cross-linking agent, and, optionally, one or more plasticisers and/or fillers;
homogenising the materials batch by mixing to form a batch mixture;
moulding the batch mixture into at least one shape;
curing the moulded shape by applying heat and/or pressure to cause cross-linking of the cross-linkable polymer by the peroxide cross-linking agent;
punching or cutting out moulded plastics components from the moulded shape after curing,
wherein the method further comprises the quality control method of claim 1.

15. The method of manufacturing a series of moulded plastics components of claim 14, further comprising modifying mixing parameters of the homogenising step and/or modifying heat and/or pressure conditions of the curing step in dependence on the measured residual peroxide content.

16. The method of manufacturing a series of moulded plastics components of claim 14, wherein the moulded plastics components are elastomeric seals, and wherein the method further comprises assembling the elastomeric seals into metering valves for pressurised metered dose canisters.

17. The quality control method of claim 4, wherein the cross-linked elastomer is ethylene propylene diene.

18. The quality control method of claim 8, wherein the solvent extraction is acetone extraction.

19. The quality control method of claim 9, wherein the chromatography is gas chromatography.

20. The quality control method of claim 2, wherein the threshold residual peroxide content is 35 μg/g or less.

21. The quality control method of claim 2, wherein the threshold residual peroxide content is 30 μg/g or less.

22. The method of manufacturing a series of molded plastics components of claim 14, wherein the batch mixture is molded into the at least one shape by extruding the batch mixture into a plurality of elongated strips.
